(19) United States
(12) Patent Application Publication
(10) Pub. No.: US 2023/0364384 A1
(43) Pub. Date: Nov. 16, 2023

(54) Systems, Medical Devices, and Methods for Controlling Stiffness of the Medical Devices

(71) Applicant: Bard Access Systems, Inc., Salt Lake City, UT (US)

(72) Inventors: Steffan Sowards, Salt Lake City, UT (US); Anthony K. Misener, Bountiful, UT (US); William Robert McLaughlin, Bountiful, UT (US)

(21) Appl. No.: 17/742,067

(22) Filed: May 11, 2022

(51) Int. Cl. A61M 25/01; A61M 25/00

(52) U.S. Cl. CPC A61M 25/0102; A61M 25/0068; A61M 25/0045; A61M 2025/0063; A61M 2025/0037

(57) ABSTRACT

Systems, medical devices, and methods for controlling stiffness of the medical devices are disclosed. For example, a system can include an elongate medical device such as a stylet and a pneumatic or hydraulic pump station. The medical device can include a tubular body with a lumen. The lumen, which terminates proximal of a distal end of the medical device, can be configured to contain a fluid. The pump station can be configured to pressurize the fluid and, thereby, stiffen at least a distal portion of the medical device. Being as the tubular body of the medical device is configured to be disposed in another lumen of another elongate medical device such as an intravenous catheter, any stiffness in the medical device (e.g., the stylet) can be imparted to the other medical device (e.g., the catheter) when disposed therein.

### BACKGROUND

Current solutions aimed at controlling stiffness in elongate medical devices such as stylets typically lack real-time control of the stiffness. Indeed, the current solutions aimed at controlling the stiffness in elongate medical devices are generally static. What is needed is real-time control of the stiffness of elongate medical devices.

Disclosed herein are systems, medical devices, and methods that address the foregoing.

### SUMMARY

Disclosed herein is a system including, in some embodiments, an elongate medical device and a fluid-pressurizing means for pressurizing a fluid. The medical device includes a tubular body and a lumen therein. The lumen, which terminates proximal of a distal end of the medical device, is configured to contain the fluid therein. The fluid-pressurizing means is for pressurizing the fluid and, thereby, stiffening at least a distal portion of the medical device under control of a user.

In some embodiments, the fluid-pressurizing means is also for depressurizing the fluid and, thereby, softening at least the distal portion of the medical device under control of the user.

In some embodiments, the fluid-pressurizing means is a pneumatic or hydraulic pump station including a pump configured to pressurize and depressurize the fluid.

In some embodiments, the pump station is configured with a processor, memory, and associated logic configured to pressurize the fluid along a continuum for a corresponding continuum of stiffness in at least the distal portion of the medical device under the control of the user.

In some embodiments, the fluid-pressurizing means is a syringe or bulb configured to pressurize and depressurize the fluid.

In some embodiments, the fluid is a gas.

In some embodiments, the fluid is a liquid.

In some embodiments, the lumen is aligned with a central axis of the medical device. The lumen being aligned with the central axis of the medical device allows substantially even pressurization when the fluid is pressurized for stiffening at least the distal portion of the medical device.

In some embodiments, the medical device further includes a signal-conducting means for conducting a signal along a length of the medical device.

In some embodiments, the signal-conducting means is a wire configured to conduct electrical signals.

In some embodiments, the signal-conducting means is an optical fiber configured to conduct optical signals.

In some embodiments, the medical device is a stylet. The tubular body of the stylet is configured to be disposed in a lumen of another elongate medical device.

In some embodiments, the other elongate medical device is an intravenous catheter.

Also disclosed herein is a medical device including, in some embodiments, a tubular body and a lumen therein. The lumen, which terminates proximal of a distal end of the medical device, is configured to contain a fluid therein. The fluid, when pressurized, stiffens at least a distal portion of the medical device under control of a user.

In some embodiments, the fluid is a gas.

In some embodiments, the fluid is a liquid.

In some embodiments, the lumen is aligned with a central axis of the medical device. The lumen being aligned with the central axis of the medical device allows substantially even pressurization when the fluid is pressurized for stiffening at least the distal portion of the medical device.

In some embodiments, the medical device further includes a signal-conducting means for conducting a signal along a length of the medical device.

In some embodiments, the signal-conducting means is a wire configured to conduct electrical signals.

In some embodiments, the signal-conducting means is an optical fiber configured to conduct optical signals.

In some embodiments, the medical device is a stylet. The tubular body of the stylet is configured to be disposed in a lumen of an intravenous catheter.

Also disclosed herein is a method including, in some embodiments, a disposing step, a coupling step, a filling step, and a pressurizing step. The disposing step includes disposing a first elongate medical device in a second lumen of a second elongate medical device. The first medical device includes a tubular body and a first lumen therein. The first lumen, which terminates proximal of a distal end of the first medical device, is configured to contain a fluid therein. The coupling step includes coupling the first medical device to a fluid-pressurizing means for pressurizing the fluid. The filling step includes filling the first lumen of the first medical device with the fluid. The pressurizing step includes pressurizing the fluid in the first lumen of the first medical device, thereby, stiffening at least a distal portion of the first medical device under control of a user.

In some embodiments, the method further includes a depressurizing step. The depressurizing step includes depressurizing the fluid in the first lumen of the first medical device, thereby, softening at least the distal portion of the first medical device under control of the user.

In some embodiments, the fluid is a gas.

In some embodiments, the fluid is a liquid.

In some embodiments, the first lumen is aligned with a central axis of the first medical device. The first lumen being aligned with the central axis of the first medical device allows substantially even pressurization when the fluid is pressurized for the stiffening of at least the distal portion of the first medical device.

In some embodiments, the first medical device is a stylet and the second medical device is an intravenous catheter.

These and other features of the concepts provided herein will become more apparent to those of skill in the art in view of the accompanying drawings and following description, which describe particular embodiments of such concepts in greater detail.

### DRAWINGS

FIG. 1 illustrates a system for controlling stiffness of an elongate medical device such as a stylet in accordance with some embodiments.

FIG. 2 illustrates the stylet disposed in another elongate medical devices such as an intravenous catheter in accordance with some embodiments.

FIG. 3 illustrates a transverse cross section of the stylet disposed in the catheter in accordance with some embodiments.

FIG. 4 illustrates a perspective view of the stylet in accordance with some embodiments.

FIG. 5 illustrates a transition of the stylet from an unpressurized state to a pressurized state with a stiffness in accordance with some embodiments.

FIG. 6 illustrates a transverse cross section of the stylet in the unpressurized state in accordance with some embodiments.

FIG. 7 illustrates a transverse cross section of the stylet in the pressurized state in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or “proximal section” of, for example, a stylet includes a portion or section of the stylet intended to be near a clinician when the stylet is used on a patient. Likewise, a “proximal length” of, for example, the stylet includes a length of the stylet intended to be near the clinician when the stylet is used on the patient. A “proximal end” of, for example, the stylet includes an end of the stylet intended to be near the clinician when the stylet is used on the patient. The proximal portion, the proximal section, or the proximal length of the stylet can include the proximal end of the stylet; however, the proximal portion, the proximal section, or the proximal length of the stylet need not include the proximal end of the stylet. That is, unless context suggests otherwise, the proximal portion, the proximal section, or the proximal length of the stylet is not a terminal portion or terminal length of the stylet.

With respect to “distal,” a “distal portion” or a “distal section” of, for example, a stylet includes a portion or section of the stylet intended to be near or in a patient when the stylet is used on the patient. Likewise, a “distal length” of, for example, the stylet includes a length of the stylet intended to be near or in the patient when the stylet is used on the patient. A “distal end” of, for example, the stylet includes an end of the stylet intended to be near or in the patient when the stylet is used on the patient. The distal portion, the distal section, or the distal length of the stylet can include the distal end of the stylet; however, the distal portion, the distal section, or the distal length of the stylet need not include the distal end of the stylet. That is, unless context suggests otherwise, the distal portion, the distal section, or the distal length of the stylet is not a terminal portion or terminal length of the stylet.

Unless defined otherwise, all technical and scientific terms used herein have the same meaning as commonly understood by those of ordinary skill in the art.

As set forth above, the current solutions aimed at controlling the stiffness in elongate medical devices such as stylets typically lack real-time control of the stiffness. Indeed, the current solutions aimed at controlling the stiffness in elongate medical devices are generally static. What is needed is real-time control of the stiffness of elongate medical devices.

Disclosed herein are systems, medical devices, and methods for controlling stiffness of the medical devices. For example, a system can include an elongate medical device such as a stylet and a fluid-pressurizing means such as a pneumatic or hydraulic pump station for pressurizing a fluid a. The medical device can include a tubular body with a lumen. The lumen, which terminates proximal of a distal end of the medical device, can be configured to contain the fluid. The fluid-pressurizing means is for pressurizing the fluid and, thereby, stiffening at least a distal portion of the medical device under control of a user. Being as the tubular body of the medical device is configured to be disposed in another lumen of another elongate medical device such as an intravenous catheter, any stiffness in the medical device (e.g., the stylet) can be imparted to the other medical device (e.g., the catheter) when disposed therein.

### Systems

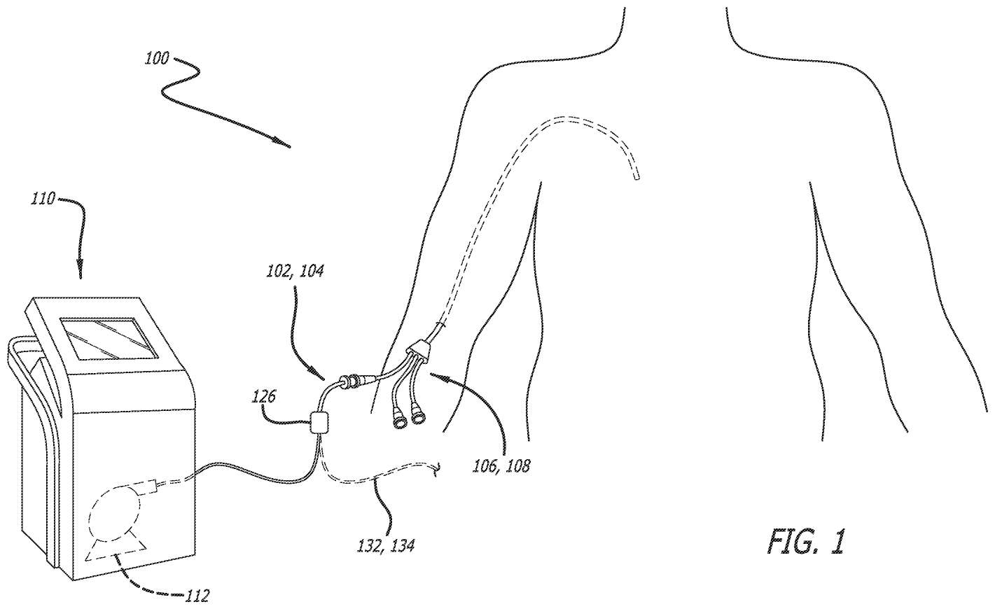

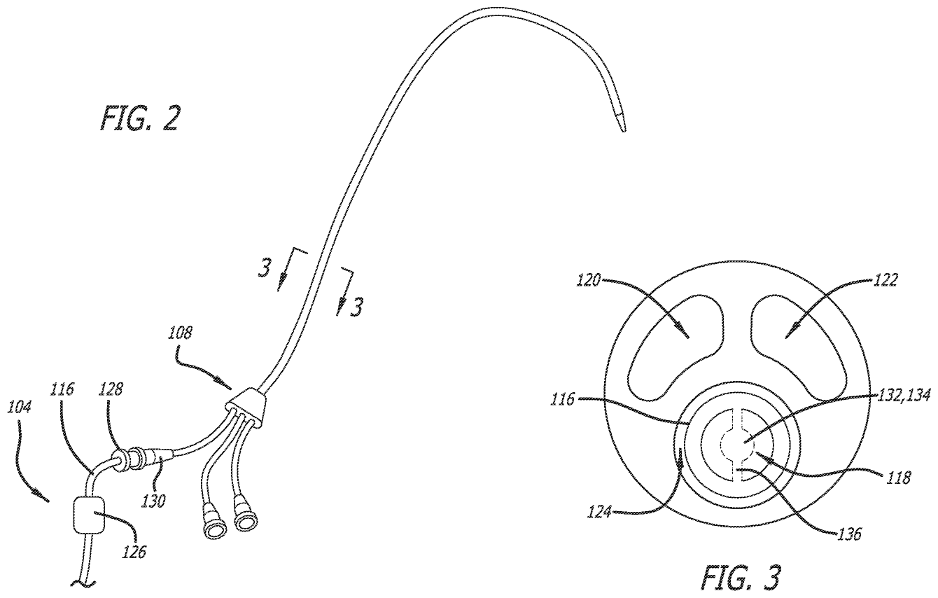

FIG. 1 illustrates a system 100 for controlling stiffness of an elongate medical device 102 such as a stylet 104 in accordance with some embodiments. FIG. 2 illustrates the stylet 104 disposed in another elongate medical device 106 such as an intravenous catheter 108 in accordance with some embodiments. FIG. 3 illustrates a transverse cross section of the stylet 104 disposed in the catheter 108 in accordance with some embodiments.

As shown, the system 100 can include a pneumatic or hydraulic pump station 110 and at least the medical device 102, for example, the stylet 104. Notably, the system 100 can also include the other medical device 106, for example, the catheter 108, into which the medical device 102 is configured to be disposed. Indeed, the tubular body 116 of the medical device 102 is configured to be disposed in the other medical device 106 to control stiffness in the other medical device 106 by controlling the stiffness in the medical device 102.

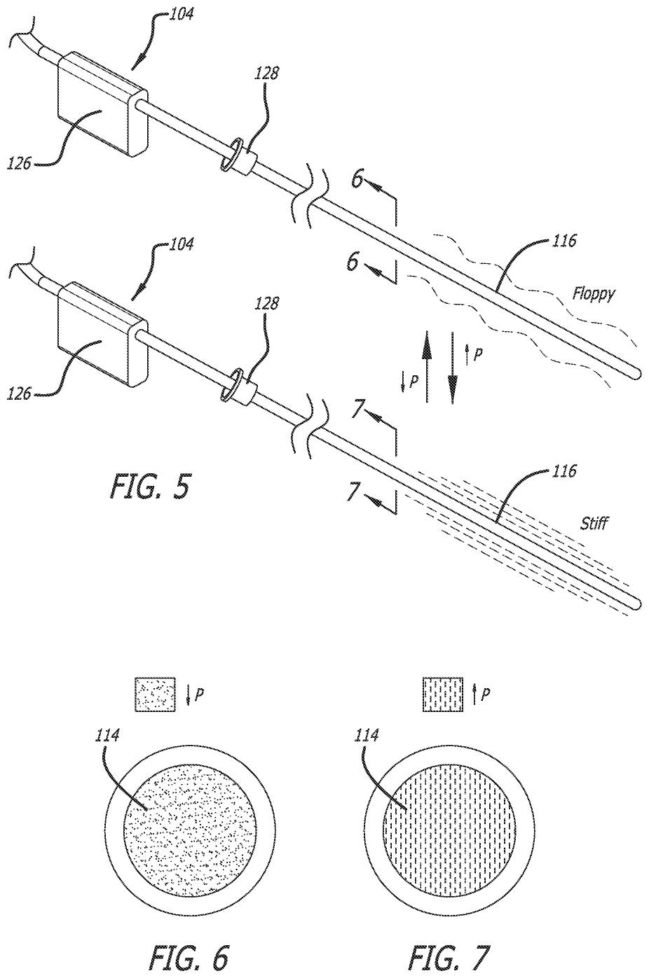

When present, the pump station 110 includes a pump 112 and an embedded system (not shown) for operating the system 100 under control of a user such as a clinician. The pump 112 is configured to pressurize a fluid 114 in the medical device 102 and, thereby, stiffen at least a distal portion of the medical device 102. The pump 112 is also configured to depressurize the fluid 114 and, thereby, soften at least the distal portion of the medical device 102. (See, for example, FIG. 5.) The embedded system can include a system on a chip, a microcontroller, or the like having a processor, memory, and associated logic configured to pressurize the fluid 114 along a continuum for a corresponding continuum of stiffness in at least the distal portion of the medical device 102. Likewise, the embedded system is configured to depressurize the fluid 114 along the continuum for the corresponding continuum of stiffness in at least the distal portion of the medical device 102.

Notably, whether the pump station 110 is present or absent, the fluid-pressurizing means can alternatively be a syringe, a bulb (e.g., a pipet bulb), or some other hand-operated pump-like device configured to pressurize and depressurize the fluid under control of the user.

The fluid 114 for which the system 100 is configured can include a gas such as nitrogen, argon, or compressed air or a liquid such as water or saline. Such a fluid can be stored in a reservoir such as a replaceable tank or canister fluidly connected to the pump 112.

### Medical Devices

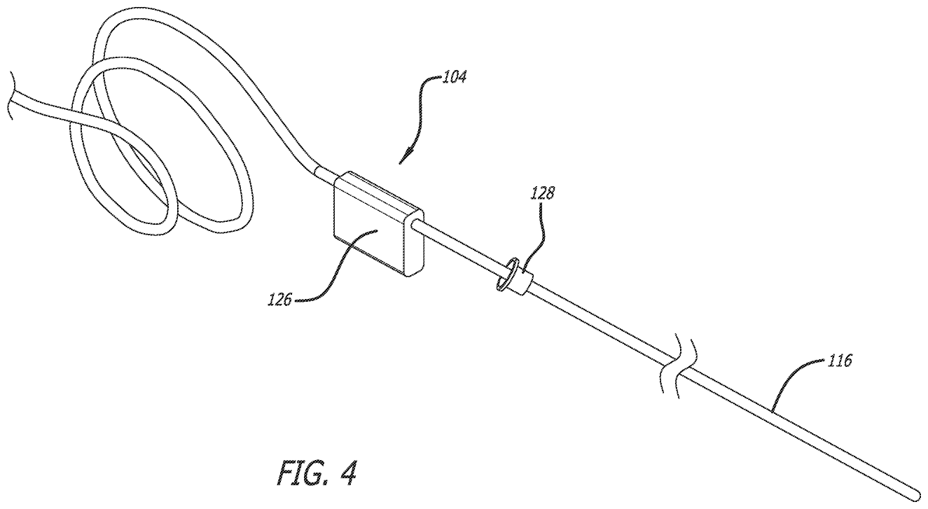

FIG. 4 illustrates a perspective view of the stylet 104 in accordance with some embodiments.

As shown, the medical device 102 can be the stylet 104. For convenience, the stylet 104 will serve as a particular species of the medical device 102 herein. However, it should be understood that the medical device 102, being the genus to which the stylet 104 belongs, includes features of the stylet 104 set forth below.

The stylet 104 includes a tubular body 116 and a lumen 118 in the tubular body 116.

The tubular body 116 of the stylet 104 is configured to be disposed in a lumen of the catheter 108 as a species of the other medical device 106 to control stiffness in the catheter 108 by controlling the stiffness in the stylet 104. Indeed, FIGS. 2 and 3 show the stylet 104 disposed in a secondary or tertiary lumen 120 or 122 of the catheter 108 for controlling the stiffness in the catheter 108. (Notably, the stylet 104 can be disposed in a primary lumen 124 of the catheter 108 for controlling the stiffness in the catheter 108 all the way to a distal tip of the catheter 108, which distal tip the secondary or tertiary lumen 120 or 122 are typically short of) In addition, the tubular body 116 of the stylet 104 can be formed of a polymer (e.g., a polyurethane), optionally, in a multilayered structure including an intervening reinforcement layer for higher pressures. While not shown, such an intervening reinforcement layer can be a braided tube of a metal or a same or different polymer than a remainder of the tubular body 116.

FIG. 5 illustrates a transition of the stylet 104 from an unpressurized state to a pressurized state with a stiffness in accordance with some embodiments. FIG. 6 illustrates a transverse cross section of the stylet 104 in the unpressurized state in accordance with some embodiments. FIG. 7 illustrates a transverse cross section of the stylet 104 in the pressurized state in accordance with some embodiments.

As shown, the lumen 118, which terminates proximal of a distal end of the stylet 104, is configured to contain the fluid 114 therein. The fluid 114, when pressurized, stiffens at least a distal portion of the stylet 104 under control of the user. Notably, the lumen 118 is aligned with a central axis of the stylet 104. The lumen 118 being aligned with the central axis of the stylet 104 allows substantially even pressurization when the fluid 114 is pressurized for stiffening at least the distal portion of the stylet 104.

The stylet 104 can further include a handle 126 around the tubular body 116 of the stylet 104 but proximal of an insertable portion of the tubular body 116. The handle 126, when present, is configured for handling the stylet 104 thereby.

The stylet 104 can further include a Luer connector 128 around the tubular body 116 of the stylet 104 configured to insert or screw into a complementary Luer connector 130 of the catheter 108. The Luer connector 128 can be slidably disposed around the tubular body 116 of the stylet 104 for adjusting a length of the stylet 104 disposed in the catheter 108.

The stylet 104 can further include a signal-conducting means for conducting a signal along a length of the medical device 102. For example, the signal-conducting means can be a wire 132 configured to conduct electrical signals such as ECG signals, or the signal-conducting means can be an optical fiber 134 configured to conduct optical signals such as those reflected back from fiber Bragg gratings (“FBGs”) in the optical fiber. Such a signal-conducting means, when present, can be freely disposed in the lumen 118 of the stylet 104, integrated (e.g., coextruded) into a luminal or abluminal wall of the stylet 104, integrated (e.g., coextruded) into a partial septum (e.g., a septum short of a luminal wall opposite that from which the septum originates), or integrated (e.g., coextruded) into a perforated but otherwise complete septum 136 as shown in FIG. 3. Notably, the perforated septum 136 includes perforations therethrough configured for fluid flow across the perforated septum 136, which allows substantially even pressurization when the fluid 114 is pressurized for stiffening at least the distal portion of the stylet 104.

### Methods

Methods include at least a method of using the system 100. Such a method can include one or more steps selected from a disposing step, a coupling step, a filling step, a pressurizing step, and a depressurizing step.

The disposing step includes disposing the stylet 104 in a lumen (e.g., the primary, secondary, or tertiary lumen 120, 122, or 124) of the catheter 108. Again, the stylet 104 includes the tubular body 116 and the lumen 118 therein. The lumen 118, which terminates proximal of the distal end of the stylet 104, is configured to contain the fluid 114 therein. And being that the lumen 118 is aligned with the central axis of the stylet 104, the lumen 118 allows substantially even pressurization when the fluid 114 is pressurized for stiffening at least the distal portion of the stylet 104.

The coupling step includes coupling the stylet 104 to the fluid-pressurizing means for pressurizing the fluid.

The filling step includes filling the lumen 118 of the stylet 104 with the fluid 114.

The pressurizing step includes pressurizing the fluid 114 in the lumen 118 of the stylet 104, thereby, stiffening at least the distal portion of the stylet 104 under control of the user.

The depressurizing step includes depressurizing the fluid 114 in the lumen 118 of the stylet 104, thereby, softening at least the distal portion of the stylet 104 under control of the user.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

What is claimed is:

1. A system, comprising:
an elongate medical device including:
a tubular body; and
a lumen terminating proximal of a distal end of the medical device, the lumen configured to contain a fluid therein; and
a fluid-pressurizing means for pressurizing the fluid and, thereby, stiffening at least a distal portion of the medical device under control of a user.

2. The system of claim 1, wherein the fluid-pressurizing means is also for depressurizing the fluid and, thereby, softening at least the distal portion of the medical device under control of the user.

3. The system of claim 2, wherein the fluid-pressurizing means is a pneumatic or hydraulic pump station including a pump configured to pressurize and depressurize the fluid.

4. The system of claim 3, wherein the pump station is configured with a processor, memory, and associated logic configured to pressurize the fluid along a continuum for a corresponding continuum of stiffness in at least the distal portion of the medical device under the control of the user.

5. The system of claim 2, wherein the fluid-pressurizing means is a syringe or bulb configured to pressurize and depressurize the fluid.

6. The system of claim 1, wherein the fluid is a gas.

7. The system of claim 1, wherein the fluid is a liquid.

8. The system of claim 1, wherein the lumen is aligned with a central axis of the medical device to allow substantially even pressurization when the fluid is pressurized for stiffening at least the distal portion of the medical device.

9. The system of claim 8, wherein the medical device further includes a signal-conducting means for conducting a signal along a length of the medical device.

10. The system of claim 9, wherein the signal-conducting means is a wire configured to conduct electrical signals.

11. The system of claim 9, wherein the signal-conducting means is an optical fiber configured to conduct optical signals.

12. The system of claim 1, wherein the medical device is a stylet, the tubular body of the stylet configured to be disposed in a lumen of another elongate medical device.

13. The system of claim 12, wherein the other elongate medical device is an intravenous catheter.

14. A medical device, comprising:
a tubular body; and
a lumen terminating proximal of a distal end of the medical device, the lumen configured to contain a fluid therein, which fluid, when pressurized, stiffens at least a distal portion of the medical device under control of a user.

15. The medical device of claim 14, wherein the fluid is a gas.

16. The medical device of claim 14, wherein the fluid is a liquid.

17. The medical device of claim 14, wherein the lumen is aligned with a central axis of the medical device to allow substantially even pressurization when the fluid is pressurized for stiffening at least the distal portion of the medical device.

18. The medical device of claim 17, wherein the medical device further includes a signal-conducting means for conducting a signal along a length of the medical device.

19. The medical device of claim 18, wherein the signal-conducting means is a wire configured to conduct electrical signals.

20. The medical device of claim 18, wherein the signal-conducting means is an optical fiber configured to conduct optical signals.

21. The medical device of claim 14, wherein the medical device is a stylet, the tubular body of the stylet configured to be disposed in a lumen of an intravenous catheter.

22. A method, comprising:
disposing a first elongate medical device in a second lumen of a second elongate medical device, the first medical device including:
a tubular body; and
a first lumen terminating proximal of a distal end of the first medical device, the first lumen configured to contain a fluid therein; and
coupling the first medical device to a fluid-pressurizing means for pressurizing the fluid;
filling the first lumen of the first medical device with the fluid; and
pressurizing the fluid in the first lumen of the first medical device, thereby, stiffening at least a distal portion of the first medical device under control of a user.

23. The method of claim 22, further comprising:
depressurizing the fluid in the first lumen of the first medical device, thereby, softening at least the distal portion of the first medical device under control of the user.

24. The method of claim 23, wherein the fluid is a gas.

25. The method of claim 23, wherein the fluid is a liquid.

26. The method of claim 23, wherein the first lumen is aligned with a central axis of the first medical device to allow substantially even pressurization when the fluid is pressurized for the stiffening of at least the distal portion of the first medical device.

27. The method of claim 23, wherein the first medical device is a stylet and the second medical device is an intravenous catheter.
